(19) United States
(12) Patent Application Publication
(10) Pub. No.: US 2004/0132092 A1
(43) Pub. Date: Jul. 8, 2004

(54) Determining the density of functional moieties on polymer reagents

(76) Inventors: Christopher M. Stetson, West Forks, ME (US); James P. Albarella, Granger, IN (US); Paul F. Corey, Elkhart, IN (US)

Correspondence Address: JENKENS & GILCHRIST, P.C., 225 WEST WASHINGTON, SUITE 2600, CHICAGO, IL, 60606, US

(21) Appl. No.: 10/336,573

(22) Filed: Jan. 3, 2003

(51) Int. Cl. G01N 33/53; G01N 33/574

(52) U.S. Cl. 435/7.1; 435/7.23

(57) ABSTRACT

The invention provides a method for determining the density of functional molecules attached to a substrate used for analysis of biological samples. A dye molecule responding to near infrared radiation at a wavelength of at least 600 nm is attached to the substrate and used to indicate the number of the functional molecules attached to the substrate by comparing the infrared absorption of the dye molecules with the ultraviolet absorption of the functional molecules. Such substrates may be employed in immunoassays and in vivo diagnostics.

### BACKGROUND OF THE INVENTION

This invention relates generally to analysis of biological samples. More particularly, the invention relates to polymer reagents which are used in various types of assays, including for example, lateral flow immunoassays, agglutination assays, and sol particle inhibition immunoassays. The polymer reagents have attached to them certain functional moieties which react with components in the sample being analyzed to indicate the condition of the person from whom the sample has been taken. Such functional moieties include, for example, haptens, epitopes, antibodies, antigens, immunoglobulins.

It is important to know how many functional moieties are attached to a polymer chain in order that an accurate analysis is obtained. Clearly, if the number of functional moieties present in a reagent is variable, then the results obtained are less precise, and more qualitative than would be desired.

If functional molecules are brought into contact with an activated polymer substrate, they will attach themselves at activated sites. The excess functional molecules should be removed in order to prevent them from reacting with a sample and producing inaccurate results. The attached molecules may be relatively few compared to the potential sites on the polymer substrate. Thus, for typical polymers it is difficult to determine the number of the potential sites which are occupied by functional molecules, which may be referred to as the density of the functional molecules. Typically, this has been done by measuring the number of attached functional moieties by ultraviolet absorption and then correcting for other factors which interfere with the measurement of the attached functional moieties, particularly the polymer substrate. Such methods involve assumptions to untangle overlapping spectra. Thus, they are inconvenient for use in commercial applications in which polymer substrates are functionalized for use in biological assays.

One potential method of determining the density of functional molecules attached to a polymer substrate is to attach a dye molecule as an indicator for the attached functional moieties. However, most dye molecules strongly absorb at wavelengths which are coincident with those of the typical functional moieties.

Cyanine dyes have been used to label various molecules, such as those used in complex biological matrices found in vivo diagnostic applications. They have been the subject of a number of patents, including U.S. Pat. No. 6,114,350; U.S. Pat. No. 6,020,867; U.S. Pat. No. 6,083,485; U.S. Pat. No. 5,650,334; U.S. Pat. No. 5,965,713; U.S. Pat. No. 5,256,542; WO 97/13810; U.S. Pat. No. 6,048,982; U.S. Pat. No. 5,627,027; U.S. Pat. No. 5,569,766; U.S. Pat. No. 5,569, 587; U.S. Pat. No. 5,486,616; U.S. Pat. No. 5,268,486; and U.S. Pat. No. 6,027,709. These dyes have been difficult to apply since they often present solubility problems in diagnostic media used for biological samples.

Various polymers have been used as carriers for functional moieties that react with components in biological samples. For example, polysaccharides, polypeptides, polyamides, polyamines, polyesters, polyethylene glycol and related copolymers. One polymer which has many potential applications is Ficoll® which results from the crosslinking of sucrose by epichlorhydrin. This polymer has a high molecular weight and is useful in analytical chemistry, where it finds applications in centrifugal cell separations and centrifugal isolation of viruses.

The present invention will be illustrated below using such sucrose polymers. In one example, the molecular weight of the polymer is in the range of 300,000 to 500,000. In order to attach molecules which serve as indicators for analytical purposes, the hydroxyl groups on the polymer are reacted with compounds which provide active sites for attachment of the molecules. In a polymer with a very high molecular weight it will be evident that many potential sites will be established. It is important for accuracy in analytical work that the number of sites which actually react with the functional molecules is measured, that is, the density of the functional moieties on the polymer. Excess functional molecules, i.e. which have not been attached to the polymer, should be removed in order that only those actually attached to the polymer substrate are measured. The present inventors have found a method of improving the accuracy of analytical procedures which employ polymer substrates for the functional moieties which react with biological samples. In their method the density of attached functional moieties is determined by use of unique dye molecules, as will be shown in the discussion below.

### SUMMARY OF THE INVENTION

The invention includes a method for determining the density of functional moieties attached to a polymer substrate used for analysis of biological samples. A dye molecule responding to near radiation at a wavelength of at least 600 nm, preferably greater than about 700 nm, most preferably above 800 nm is attached to the substrate as a reference label indicating the amount of the substrate molecules containing the functional moieties. Comparing the absorption of radiation by the functional moieties with the absorption of the dye molecules, the density (the concentration) of the functional molecules on the substrate can be determined.

Dye molecules useful in the invention include cyanine dyes, preferably those having the formula:

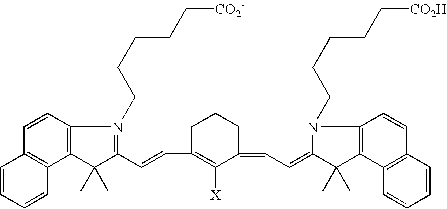

where: X=S(CH₂)₂SO₃H, S(CH₂)₆, SCH₃, S(CH₂)ₙ, SR, NH₂, NHY, N₃, I, Cl, Br

n=1-12

R=cyclohexane, isopropyl, isobutyl

Y=CH₃, (CH₂)ₘ, CH₃ and m is 1-11

In one preferred embodiment the polymer substrate is aminoethylcarbonylmethyl ficoll (AECM Ficoll®) and the dye molecule is DTO-108, one of the indocyanine dyes within the above formula where X=S(CH₂)₂SO₃H.

Functional moieties include those active in molecular recognition, including haptens, epitopes, antibodies, antigens and immunoglobulins.

In another embodiment, the invention is a group of immunoassays in which polymers labeled with the cyanine dyes are used to determine the density of functional moieties attached to analytes in biological samples. For example, the labeled polymers may be used for determining analyte concentration in spectrally complex media such as blood, feces, dark urine and other opaque body fluids. In another embodiment, in vivo diagnostics may achieve improved imaging by employing the polymers labeled with the cyanine dyes.

### BRIEF DESCRIPTION OF THE DRAWINGS

FIG. 1 is an UV spectrum of a functionalized polymer substrate from the Example.

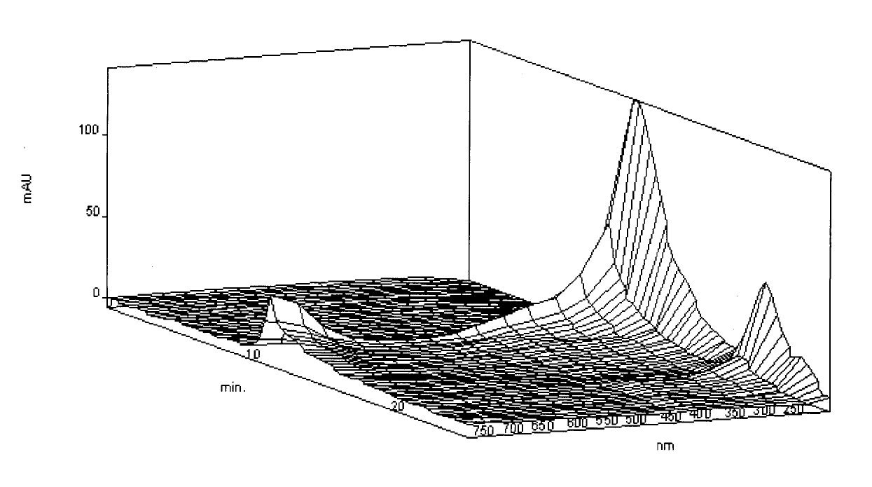

### DESCRIPTION OF THE PREFERRED EMBODIMENTS

Functional Moieties

It is expected that useful functional molecules which would be attached to substrates and used for analysis of biological samples would include those considered active in molecular recognition, for example, haptens, epitopes, antibodies, antigens, immunoglobulins, and the like. Typically, such functional molecules absorb radiation in the range of 200 to 600 nm and consequently often fall within the region occupied by the polymer substrates. Thus the amount of the functional moieties present in the substrate is difficult to measure. Examples of such functional molecules include haptons, epitopes, antibodies, antigens, immunoglobulins and the like. In the example below, Cytochrome C, a model protein, was chosen to illustrate the invention since it has convenient optical properties. Cytochrome C is an essential component of the mitochondrial respiratory chain and its release during apoptotic cell death makes it a useful research probe.

Substrates

Typical high molecular weight polymers have a distribution of molecular weights about an average value, which may vary from sample to sample. If a functional molecule is to be attached to the polymer the amount which reacts with the polymer may vary. Thus, one cannot assume that a given amount of the functional molecule would react with a given amount of a polymer substrate. Furthermore, the activity of the chemically activated polymer may vary also, so that after one reacts a functional molecule with a polymer and then removes the excess reactants, it is uncertain how much of the functional molecule has been attached to the polymer and determining activity, that is, affinity and functionality has been found to be difficult and time consuming.

Measurement of the ultraviolet absorption of the functionalized polymer might be used to determine how much of the functional molecule has been attached. There are difficulties to be overcome if accurate results are to be obtained by this method, although some of these difficulties can be circumvented. Typical polymer substrates absorb ultraviolet radiation in the general range of about 200-350 nm. Since the amount of the polymer is large relative to the amount of the functional molecules, the observed peak in an ultraviolet scan of the polymer will often overlap with the peak characteristic of the functional molecule. Therefore, in determining the amount of the functional molecule associated with each polymer molecule, it is necessary to measure the polymer separately to provide a base for determining the effect of the functional molecules. This is an approximate, inconvenient procedure and existing methods are not well suited to quality control in commercial applications. As previously mentioned, the polymers also may vary from batch to batch and thus it should not be assumed that the baseline measurements of the polymers are always the same.

If the polymers could be labeled with an infrared absorbing dye, then a comparison of the infrared absorption of the label molecule with the ultraviolet spectrum of attached functional moieties could be used to indicate the amount of the functional molecule which had been attached to the polymer molecules. Unfortunately, most dye molecules absorb ultraviolet radiation within the same region as the functional moieties and therefore cannot provide a clear measure of the polymer molecules, since they also will contribute to the absorption of the polymer and the functional molecule. Potentially, a dye molecule which absorbs infrared radiation at a longer wavelength than the functional moieties and independently measurable from their ultraviolet spectrum could be used. These are uncommon, but the application of such a system is described herein.

In the example below, a modified Ficoll® is used as a substrate. It has the advantage of being very soluble and thus improving the solubility of the selected near infrared indocyanine dye molecules, which typically are not very soluble in aqueous mixture. Ficoll is sucrose which has been crosslinked with epichlorohydrin. It has many uses in centrifugal cell and virus separations.

Of particular interest in the present invention is a modified Ficoll®, aminoethylcarbonylmethyl ficoll, (AECM Ficoll), which is prepared by reacting the sucrose polymer with chloroacetic acid and diaminoethane. Unmodified Ficoll is commercially available from Amersham Bioscience. The functionalized Ficoll is especially suited to attaching dye molecules and functional moieties such as those mentioned above. The functionalized Ficoll is relatively transparent to ultraviolet radiation compared to most polymer substrates and absorbs ultraviolet radiation up to 250-260 nm. That is close to the region in which are found many functional moieties used in analysis of biological samples, typically 275 nm. This makes it difficult to separate the ultraviolet absorption of the functional moiety of interest from that of the substrate. For example, once a protein is attached to a Ficoll molecule, the spectrum of the protein can be discerned and the amount of the protein can be estimated. But, the amount of polymer and the amount of protein attached to a Ficoll molecule cannot be conveniently or accurately determined.

In addition to the AECM Ficoll, other polymer substrates may have application in the invention, for example, polyacrylic acid, polyamides, polypeptides, dextrans, polyesters, polyethylene glycols, polyamines, and co-polymers thereof.

Labeling Polymer Mixtures

One problem which arises when one attempts to locate a suitable dye molecule for labeling polymers has already been mentioned. Many dyes absorb strongly at wavelengths which are too close to the absorbance of the polymers and the usual functional molecules to determine accurate ratios. Dyes will often washout the UV spectrum of the polymer-functional molecule conjugate. This requires cumbersome analytical procedures in order to separate the respective effects of the polymers, the functional moieties, and the dye molecules and the results are considered to be inaccurate.

While some dye molecules can be used as just described, they have been difficult to apply since they have limited solubility. The present invention uses unique indocyanine dye molecules which can be reacted with polymers and which absorb at a wavelength outside the range of the functional moieties and polymers typically used for biological assays. With the use of such a dye molecule, and related dye molecules having similar characteristics, it becomes possible to label polymer molecules and then compare the ultraviolet absorption of a functional molecule attached to the polymer with the infrared absorption of the dye labeled polymer, without a need to establish the baseline absorption characteristic of the polymer each time. Instead, the dye serves as a reference since it characterizes the polymer.

The dye molecule of particular interest is designated DTO-108, which is a indo cyanine dye having the following molecular structure:

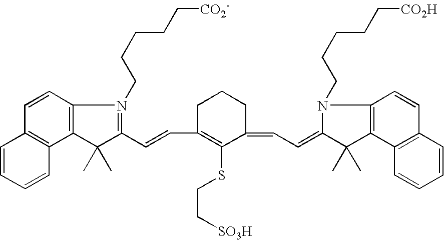

The molecule can be prepared from a reaction between a precursor disclosed in Lee et al. U.S. Pat. No. 5,453,505 and mercaptoethane sulfuric salt according to the method disclosed in Harada et al. U.S. Pat. No. 5,445,930. The dye molecule has a characteristic ultraviolet absorption peak at about 878 nm, although experience suggests that absorption at about 850 nm occurs when the dye is conjugated with AECM Ficoll and observed in water.

Once the value of DTO-108 has been recognized, it is expected that other dye molecules having strong absorbance above 600 nm, preferably above 700 nm, most preferably above 800 nm should be useful, provided that they can be attached to the polymer substrate of choice. In general, dyes related to DTO-108, more generally defined by the formula:

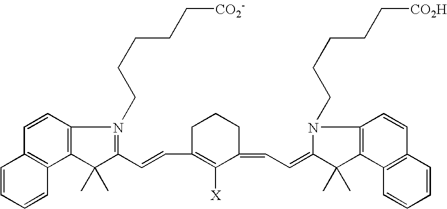

Where: X=S(CH₂)₂SO₃H, S(CH₂)₆, SCH₃, S(CH₂)ₙ, SR, NH₂, NHY, N₃, I, Cl, Br

n=1-12

R=cyclohexane, isopropyl, isobutyl

Y=CH₃, (CH₂)ₘCH₃ and m is 1-11

may be used, although not necessarily with the same results as provided by DTO-108. There is an advantage to dye molecules that are sufficiently soluble so that they can be efficiently attached to polymers in aqueous solution. Certain polymers such as the sucrose polymer used in the example below are very soluble in water and therefore they render the dye molecules very soluble as they are attached to the polymer. Alternatively, a polymer could be suspended in an organic solvent, reacted with dye molecules, and used in aqueous compositions.

In the example below, the hydroxyl groups on sucrose chains are attached to the dye molecule by reaction with the amine groups on the AECM Ficoll described above, although other methods could be used, such as through sulfhydryl, activated carboxyl groups or displacement of leaving groups such as chloride, bromide, and iodide.

### EXAMPLE

I. Activating DTO-108

To 2.0 mg of DTO-108 (produced by one of the inventors) in 10 mL of dry acetonitrile was added 7.3 mg of 1,1′-carbonyl diimidazole (“CDI”) as a solid (Sigma-Aldrich) and the mixture was stirred for 30 minutes at room temperature using sonication. Then, the acetonitrile was removed by Rotovap (bath temperature 40° C. max) and 6.0 mL of a pH 7.5, 100 mM phosphate buffer was added. The activated DTO-108 was then a 0.33 mg DTO-108/mL solution.

II. Labeling Ficoll

20 mg of aminoethylcarbonylmethyl ficoll (AECM ficoll) was dissolved in 1 mL of 100 mM pH 7.5 phosphate buffer. Then, 1.32 mL of the activated DTO-108 solution was added to the AECM ficoll solution and stirred overnight at room temperature. The solution was lyophilized to a powder, which was dissolved in 1 mL of deionized water. The aqueous solution was added to a G-100, 1.8×40 cm Sephadex column and eluted with deionized water. Material eluted between 27.65 and 46.03 minutes was collected. Of this, 27.89 mg of solids containing ficoll labeled with DTO-108 were obtained by lyophilizing the selected eluted fractions. Calibration of the DTO-108 was done using a 0.01 mg/mL solution in methanol. The concentration of DTO-108 in the ficoll was calculated to be 3.15×10⁻⁹ mols compared to 2.49×10⁻⁹ mols of ficoll. Thus, it was concluded that there were about 1.3 molecules of DTO-108 for each ficoll molecule on the average.

III. Reacting Ficoll Labeled with DTO-108 with Cytochrome C

The AECM ficoll labeled with DTO-108 described above was linked to the functional substituent Cytochrome C (Sigma-Aldrich) by reaction with PEG (polyethylene glycol) and carbonyl diimidazole. To 5.0 mg of AECM ficoll+DTO-108, 500 μL of 100 mM phosphate buffer pH 7.5 was added 22.4 mg of biscarbonyl imidazole polyethylene glycol, MW 3400 (CDI 3400 from Shearwater Polymers) in 200 μL of 100 mM phosphate buffer pH 7.5. The mixture was allowed to react for 4 hours at room temperature. Then, the reaction mixture was passed into a 1.8×40 cm Sephadex G-100 column equilibrated with pH 7.0, 1.5 mM NaCl, 1 mM phosphate buffer. Elution progress was monitored by UV and conductance. UV monitor was of the Isco, Inc. type. Conductance was monitored by an electrode and YSI monitor in the 200 μohm range. Flow rate was controlled by peristalic pump at 0.5 mL/min.

IV. Reaction with Cytochrome C

The first two fractions exiting the column were pale green and were found to contain the desired product. These fractions were frozen and lyophilized to yield a greenish powder, which was then added to 500 μL of pH 7.5, 100 mM phosphate buffer. To this solution was added 1.5 mg of Cytochrome C in 200 μL pH 7.5 phosphate buffer. After allowing the reaction to proceed overnight at room temperature, the crude mixture was applied to a Sephadex G-200 column 18×40 cm. The product was collected in two fractions and combined, yielding about 2.4 mL. The ultraviolet absorption at 408 nm for Cytochrome C and 850 nm for DTO-108 was used to calculate the concentration of DTO-108 to be 4.51×10⁻⁷ and the Cytochrome C to be 3.89×10⁻⁷ mol. It was concluded that about 0.86 molecules of Cytochrome C were attached to each molecule of ficoll. It was concluded that for each mole of ficoll, about 1.3 mol of DTO-108 and about 0.86 mol of Cytochrome C were attached. The figure illustrates the results. It can be seen that DTO-108 is attached to the AECM Ficoll (at 850 nm) as is the Cytochrome C (at 408 nm).

Applications of the Invention

The methods described have many applications, particularly in, but not limited to, immunoassays. For example, the labeled polymers may be used for determining analyte concentration in spectrally complex media such as blood, feces, dark urine and other opaque body fluids.

The labeled polymers of the invention may also be employed to provide improved imaging when used for in vivo diagnostics, such as for tumors and lesions. The dye molecules would be attached to the body tissues of interest through antibodies placed on the polymer substrate, the antibodies having been chosen to react with antigens found on the tissue being inspected.

1. A method of determining the density of functional moieties on polymer substrates comprising:
(a) attaching a dye molecule to a polymer substrate, said dye molecule absorbing infrared radiation at a wavelength of at least 600 nm;
(b) attaching a compound as a functional moiety to said polymer substrate with attached dye molecules, said compound being selected to react with an analyte in a biological sample;
(c) attaching the dye molecule selected in (a) as a label to a second sample of said polymer substrate and determining by their absorption of infrared radiation the number of dye molecules attached to said polymer substrate;
(d) determining the absorption of ultraviolet radiation by the functional moieties and by infrared radiation absorption of dye molecules in the polymer substrate of (b); and
(e) determining the number of functional moieties on said polymer substrate of (b) relative to the number of attached dye molecules determined in (c), said number of functional moieties representing the density of said functional molecules.

2. A method of claim 1 wherein said dye molecule has the formula: 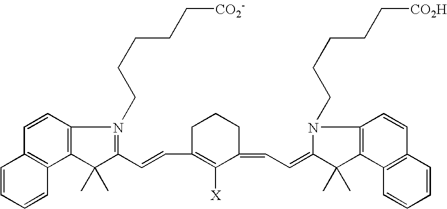
where: X=S(CH₂)₂SO₃H, S(CH₂)₆, SCH₃, S(CH₂)ₙ, SR, NH₂, NHY, N₃, I, Cl, Br
n=1-12
R=cyclohexane, isopropyl, isobutyl
Y=CH₃, (CH₂)ₘ, CH₃ and m is 1-11

3. A method of claim 1, wherein said dye molecule is DTO-108.

4. A method of claim 1 wherein said polymer substrate is a member of the group consisting of polyacrylic acid, polyamides, polypeptides, dextrans, polyesters, polyethylene glycols, polyamines, and co-polymers thereof.

5. A method of claim 4 wherein said polymer substrate is AECM Ficoll.

6. A method of claim 5 wherein said dye molecule is attached to said polymer substrate by reaction with amine functional groups on said AECM Ficoll.

7. A method of claim 6 wherein said functional compound is Cytochrome C.

8. A method of claim 7 wherein said Cytochrome C is attached to said polymer substrate by reaction with biscarbonyl imidazole terminated polyethylene glycol.

9. A method of claim 1 wherein said functional molecule as selected from the group consisting of haptens, epitopes, antibodies, antigens and immunoglobulins.

10. A method of carrying out immunoassays wherein a polymer substrate attached to a functional molecule and a dye molecule having the density of said functional molecules determined by the number of attached dye molecules is used as a reagent for determining analytes of interest in biological samples.

11. A method of claim 10 wherein said biological samples are opaque body fluids.

12. A method of claim 11 wherein said biological samples are blood, feces, and dark urine.

13. A method of carrying out in vivo diagnostics wherein a polymer substrate is used as a reagent for imaging of predetermined species of body cells, said polymer substrate being attached to a functional molecule and a dye molecule and the density of said functional molecules is determined by the number of attached dye molecules.

14. A method of claim 13 wherein said predetermined species of body cells are tumors or lesions.

15. An immunoassay wherein a functional moiety is attached to a polymer substrate and thereafter contacted with a biological sample for binding said functional moiety to an analyte, comprising:
(a) attaching a dye molecule to a polymer substrate, said dye molecule absorbing infrared radiation at a wavelength of at least 600 nm;
(b) attaching a functional moiety to said polymer substrate having an attached dye molecule of (a), said functional moiety being selected to react with an analyte in a biological sample;
(c) attaching the dye molecules of (a) as a label to a second sample of said polymer substrate and determining by their absorption of radiation the number of dye molecules attached to said polymer substrate;
(d) determining the absorption of radiation by the functional moieties and dye molecules in the polymer substrate of (b);
(e) determining the number of functional moieties in said polymer substrate of (b) relative to the number of attached dye molecules determined in (c).
(f) contacting with said biological sample the polymer substrate of (b) having said functional moiety and said dye molecule attached under suitable conditions to react said functional moiety with said analyte;
(g) measuring the absorption by radiation of the combined polymer substrate and biological sample of (f); and
(h) determining the amount of the analyte which is bound to the functional moiety by the amount of said dye molecule present.
